(19) United States
(12) Patent Application Publication
(10) Pub. No.: US 2009/0260633 A1
(43) Pub. Date: Oct. 22, 2009

(54) Selectively transparent phototherapy shades

(75) Inventor: Hendrik J. Vreman, Los Altos, CA (US)

Correspondence Address: LUMEN PATENT FIRM, 350 Cambridge, Suite 100, PALO ALTO, CA, 94306, US

(73) Assignee: The Board of Trustees of the Leland Stanford Junior University

(21) Appl. No.: 12/384,607

(22) Filed: Apr. 6, 2009

Related U.S. Application Data
(60) Provisional application No. 61/124,719, filed on Apr. 17, 2008.

(51) Int. Cl. A61F 9/00

(52) U.S. Cl. 128/858; 2/12

(57) ABSTRACT

Eye shades to reduce phototherapy light exposure to the eyes of a jaundiced person during a phototherapy session are provided. The shades includes a flexible and soft frame that can be placed against the face of a jaundiced person. At least one light-absorbing filter capable of filtering phototherapy light to both eyes of the person is affixed to the frame. The light-absorbing filter is a light intensity reduction filter of the blue-to-yellow light or the blue light from the phototherapy light. Advantages of the phototherapy shades presented herein exist due to the ability of patients to see their surroundings during phototherapy and caregivers to observe newborn's state of alertness.

### CROSS-REFERENCE TO RELATED APPLICATIONS

This application claims priority from U.S. Provisional Patent Application 61/124,719 filed Apr. 17, 2008, which is incorporated herein by reference.

### FIELD OF THE INVENTION

The invention relates generally to eye shades for phototherapy. More particularly, the invention relates to partially transparent eye shades or filters for infants undergoing phototherapy.

### BACKGROUND OF THE INVENTION

Phototherapy is commonly administered to patients or infants, particularly newborns who have hyperbilirubinemia (jaundice). However, phototherapy patients risk exposing their eyes to potentially dangerous, intense blue to yellow phototherapy light with spectral irradiance of up to 100 μW/cm²/nm. There exists evidence that consequences of bright blue light exposure can include retinal damage or changes to the eyes.

Presently, commercially available and caregiver-improvised devices with opaque or black shades are used during phototherapy. The opaque shades block out essentially all visible light. There exist reports citing the negative effects of light deprivation, such as decrease in visual acuity, functional and genomic changes, effects on color perception, development of visual and spatial attention, and developmental regulation of primary visual cortex. In addition, the opaque shades can cause irritation to the infant wearing the shades due to the inability of the infant to see its surroundings and/or caregivers. This irritation could motivate the infant to attempt to and possibly succeed at removing the shades. Furthermore, a caregiver is unable to see the infant's eyes when the opaque shades are worn; preventing the caregiver from determining if the infant is awake and ready for interaction. Accordingly, there is a need in the practice of phototherapy to develop eye shades to overcome at least some of these shortcomings.

### SUMMARY OF THE INVENTION

The present invention provides phototherapy eye shades to reduce phototherapy light exposure to the eyes of a jaundiced person during a phototherapy session. The shades includes a flexible and soft frame that can be placed against the face of a jaundiced person. At least one light-absorbing filter capable of filtering phototherapy light to both eyes of the person is affixed to the frame. The light-absorbing filter is a light intensity reduction filter of the blue-to-yellow light or the blue light from the phototherapy light.

In one example, the light-absorbing filter filters wavelengths between 400 nm and 610 nm, and the filter transmits wavelengths greater than 610 nm. The filter transmits about 2% to 20% of the blue-to-yellow light or about 5% to 20% of the blue-to-yellow light. The filter transmits at least 80% of wavelengths greater than 610 nm. In another example, the filter transmits about 2% to 20% of the blue light or about 5% to 20% of the blue light. The filter transmits about 90% of the visible light other than the blue light.

Advantages of the phototherapy shades presented herein exist due to the ability of patients to see their surroundings during phototherapy. For instance, because a newborn can see its surroundings, the eye shades need not be removed during care periods, such as feeding, breastfeeding, diaper changes, medical administration, etc. Furthermore, not only can the infant see its environment, the caregiver can also determine if the infant is awake, has its eyes open, and is ready for interaction. The ability to interact opens the potential for parental bonding which has been absent when opaque shades are used.

### BRIEF DESCRIPTION OF THE FIGURES

The present invention together with its objectives and advantages will be understood by reading the following description in conjunction with the drawings, in which:

FIG. 1 shows a front view of a phototherapy eye shade according to an embodiment of the present invention with a light-absorbing filter 110 capable of filtering phototherapy light to both eyes of a person wearing the shades. Light-absorbing filter 110 is a flexible light intensity reduction filter of the blue-to-yellow light or the blue light from the phototherapy light. Openings 120 (two on each side in this example) can be part of the filter and used for affixing a headband (not shown).

FIG. 2 shows a three-dimensional front-side view of a phototherapy eye shade according to an embodiment of the present invention including a frame 130 made out of a soft-frame material that will be placed against the person's face when the shades are worn. In this example, the frame is affixed to the back-side of the light-absorbing filter 110.

FIG. 3 shows a three-dimensional back-side view of a phototherapy eye shade according to an embodiment of the present invention.

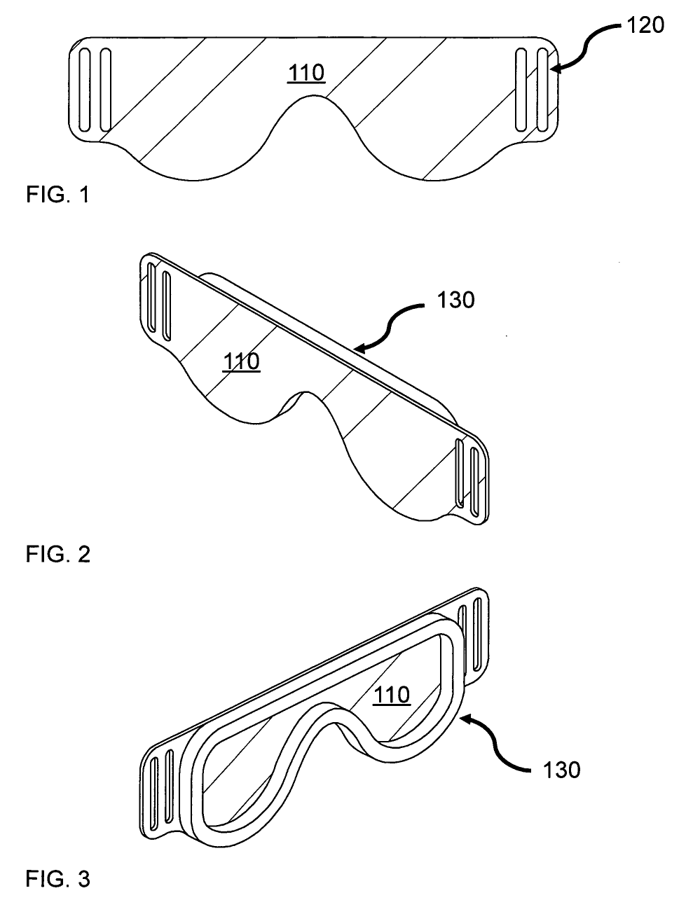

### DETAILED DESCRIPTION

The present invention is directed to shading and protecting the eyes of a patient receiving phototherapy, such as a newborn undergoing phototherapy treatment for jaundice, while simultaneously allowing the patient to visually interact with the environment. More particularly, the present invention allows for the attenuation of the intensity of phototherapy light directed, usually from overhead, at the eyes, to safe levels.

To meet these objectives, a light absorbing filter material is provided that can include any partially transparent material or combinations of materials, such as polyester, polycarbonate, styrene, elastomeric, Roscolux material, a light reflecting material such as metalized polyester films, or a nano-ceramic material such as metal oxinitride.

In an embodiment, partially and selectively transparent yellow-to-red filter shades are used to reduce the intensity of the blue-to-yellow light (400-610 nm) received by the eyes during phototherapy to approximately 2-to-20% transmission of that blue-to-yellow light delivered to the filter. In other embodiments the transmission of the blue-to-yellow light to the filter is about 3-20%, 4-20%, 5-20% or 10-20%. Wavelengths greater than 610 nm (all colors but blue-to-yellow) pass largely unimpeded (approximately 80% transmission) through the filter, which allows infants to see their surroundings, including their caregivers, and vice versa. Filters with different wavelength ranges can also be used.

In another embodiment, metalized polyester film material is used for shading during phototherapy. Silver material allows passage of approximately 90% of the visible light, but only approximately 5-to-20% of the delivered blue light. In other embodiments the transmission of the blue light to the filter is about 2-20%, 3-20%, 4-20% or 10-20%. Other metal shades, such as gray, bronze, and gold, can also be used.

The filter can be attached or be part of a soft-frame filter shades or a goggle-type device. The filter can be affixed to the distal side of, or integral to a newborn face-friendly frame. The frame can be fashioned from any soft, non-allergic material, including fabric, foam, elastomeric, combination thereof, etc. Conversely, the filter material itself may be molded to serve as an acceptable contact material to the skin surrounding the eye, while also providing the filter in front of the eyes. Prior to phototherapy, the filter and frame can be placed over the eyes of the patient and can be preferably held in place with hook/loop patches to a headband wearable behind the head of the patient. Alternatively, the frame could be held in place by hydrogel adhesive patches to be affixed to the sides of the patient's head. It is noted that any mechanisms can be used to hold the filter-containing frame over the eyes of a patient.

It is important to note that the filter glasses or goggles can be designed to be “one-sized fits all” for a wide range of infants. The filter glasses or goggles can be made at least partially of elastomeric material. The ability of the elastomeric material to stretch up to approximately 200% allows for the “one-sized fits all” design. The design can include a single lens located over both eyes. The shades and/or glasses can be made from a mold. In an exemplary embodiment, the frame and shades and headband are fabricated from a single mold. The frame is designed to fit small to large infants. The headband is designed to stretch and comfortably hold the frames to the head of small to large infants. In one embodiment, the headband is transparent to blue light so that the body area or skin underneath the headband is able to receive the phototherapy light including most if not all of the blue light.

Alternatively, the goggles may be constructed from a filtering material attached to a soft rim such as an open-cell foam and the lateral sides attached to a head-band. The open-cell foam forming the rim of the goggles will allow the passage of air into and out of the space between the eyes and the filter. Thus, two things are accomplished. First, the air immediately adjacent to the eyes is the same as the ambient room air. Second, if the goggles accidently slip over the baby's mouth and nose, the air can easily pass into and out of the space immediately adjacent to the mount and nose to prevent suffocation.

As one of ordinary skill in the art will appreciate, various changes, substitutions, and alterations could be made or otherwise implemented without departing from the principles of the present invention. Accordingly, the scope of the invention should be determined by the following claims and their legal equivalents.

What is claimed is:

1. Phototherapy eye shades, comprising a frame sized to be wearable on a person's head, wherein said frame comprises at least one light-absorbing filter capable of filtering phototherapy light to both eyes of said person and wherein said light-absorbing filter is a light intensity reduction filter of the blue-to-yellow light or the blue light from said phototherapy light.

2. The phototherapy eye shades as set forth in claim 1, wherein said light-absorbing filter filters wavelengths between 400 nm and 610 nm.

3. The phototherapy eye shades as set forth in claim 1, wherein said light-absorbing filter transmits about 2% to 20% of said blue-to-yellow light or about 5% to 20% of said blue-to-yellow light.

4. The phototherapy eye shades as set forth in claim 1, wherein said light-absorbing filter transmits wavelengths greater than 610 nm.

5. The phototherapy eye shades as set forth in claim 1, wherein said light-absorbing filter transmits wavelengths greater than 610 nm, wherein said transmission is at least 80%.

6. The phototherapy eye shades as set forth in claim 1, wherein said light-absorbing filter transmits about 2% to 20% of said blue light or about 5% to 20% of said blue light.

7. The phototherapy eye shades as set forth in claim 1, wherein said light-absorbing filter transmits about 90% of the visible light other than said blue light.

8. The phototherapy eye shades as set forth in claim 1, wherein said light-absorbing filter comprises a silver material, a gray material, a bronze material, a gold material, a polycarbonate, a polyester, a styrene, an elastomeric, a Roscolux material, a light reflecting material or a metalized polyester film.

9. The phototherapy eye shades as set forth in claim 1, wherein said frame is a single piece of material or made of a stretchable material.

10. The phototherapy eye shades as set forth in claim 1, wherein said frame fits around the head of said person.

11. The phototherapy eye shades as set forth in claim 1, wherein said frame is a one-size fits all children head sizes.

12. A method of reducing phototherapy light exposure to the eyes of a jaundiced person during a phototherapy session, comprising the step of having said jaundiced person wear a frame during said phototherapy session, wherein said frame comprises at least one light-absorbing filter capable of filtering phototherapy light to both eyes of said person and wherein said light-absorbing filter is a light intensity reduction filter of the blue-to-yellow light or the blue light from said phototherapy light.

13. The method as set forth in claim 12, wherein said light-absorbing filter filters wavelengths between 400 nm and 610 nm.

14. The method as set forth in claim 12, wherein said light-absorbing filter transmits about 2% to 20% of said blue-to-yellow light or about 5% to 20% of said blue-to-yellow light.

15. The method as set forth in claim 12, wherein said light-absorbing filter transmits wavelengths greater than 610 nm.

16. The method as set forth in claim 12, wherein said light-absorbing filter transmits wavelengths greater than 610 nm, wherein said transmission is at least 80%.

17. The method as set forth in claim 12, wherein said light-absorbing filter transmits about 2% to 20% of said blue light or about 5% to 20% of said blue light.

18. The method as set forth in claim 12, wherein said light-absorbing filter transmits about 90% of the visible light other than said blue light.
